(19) United States
(12) Patent Application Publication
(10) Pub. No.: US 2008/0311590 A1
(43) Pub. Date: Dec. 18, 2008

(54) Portable Materials and Methods for Ultrasensitive Detection of Pathogen and Bioparticles

(76) Inventors: Weihong Tan, Gainesville, FL (US); Shelly John Mechery, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/994,962

(22) PCT Filed: Oct. 10, 2006
(86) PCT No.: PCT/US06/39536

§ 371 Date: Aug. 2, 2008

Related U.S. Application Data
(60) Provisional application No. 60/725,023, filed on Oct. 7, 2005.

(51) Int. Cl. G01N 33/53; C12M 1/34; B01J 19/00; G01N 21/00; G01N 21/76

(52) U.S. Cl. 435/7.2; 435/287.1; 422/68.1; 422/82.05; 435/288.7; 435/287.2; 356/338

(57) ABSTRACT

The present invention provides systems for ultrasensitive detection of pathogens and bioparticles. One embodiment of the system comprises an optical detection scheme that allows for the detection of the fluorescence signal of bacteria or other bioparticles in less than about 20 minutes. A microflow channel allows for an assay probing volume of as little as a few picoliters. In one embodiment, the system uses RuBpy dye-doped silica nanoparticles bioconjugated with specific monoclonal antibodies of the target bioparticles. The system allows for the rapid and highly sensitive and specific detection of bacteria or other bioparticles without the need for amplification or enrichment of the sample.

### GOVERNMENT SUPPORT

The subject invention was made with government support tinder NIH Grant No. GM-66137, NIH Grant No. NS-045174 and NSF Grant No. EF-0304569. The government has certain rights in the invention.

### BACKGROUND OF THE INVENTION

The rapid and accurate detection of trace amounts of organisms such as pathogenic bacteria is important in food safety, clinical diagnosis, and military/civilian warfare. Recently, there has been much interest in the identification of various microorganisms due to the increased risks of terrorism via biological warfare agents. Escherichia coli O157:H7 (E. coli O157:H7) is one of the most dangerous food borne bacterial pathogens. It is commonly found in raw beef, fruits, vegetables, salad bar items, salami, and other food products. Outbreaks of E. coli O157:H7 infections have caused serious illnesses and led to a significant number of deaths. Therefore, in order to prevent accidental outbreaks or intentional terrorist acts, early detection of trace amounts of E. coli O157:H7 as well as other pathogenic microorganisms is critical.

The key requirements for a detection technique to be used for the early detection of microorganisms are specificity, speed, and sensitivity. Conventional detection methods provide qualitative and quantitative information in the presence of substantial amounts of organisms such as bacterial species. However, time constraints and ease of-on-site analysis are major limitations because many of these methods rely on the ability of microorganisms to grow into visible colonies over time in special growth media, which may take about 1-5 days. Moreover, detection of trace amounts of bacteria typically requires amplification or enrichment of the target bacteria in the sample. These methods tend to be laborious and time consuming because of the complicated assay procedures.

Recently, attempts have been made to improve conventional bacterial detection methods to reduce the assay time. One of these efforts has been in the modification and automation of conventional methods. In addition, many developments have evolved to improve detection techniques; for example: direct epiluminescent filter technique (DEFT), mass spectrometry-based methods, and counting and identification test kits. One of the most promising techniques is flow cytometry, which is able to detect 10²-10³ E. coli O157:H7 cells/mL within 1 hour based on luminescence signal in a flow system. Though the detection time is dramatically reduced, an improved sensitivity is still a challenge.

Thus, a need remains for a flow cytometry system with improved sensitivity that is quick, inexpensive, accurate and simple to use.

### BRIEF SUMMARY OF THE INVENTION

The present invention provides systems for ultrasensitive detection of pathogens and bioparticles. Advantageously, the systems of the subject invention are simple and can be made portable.

In one embodiment, the present invention provides a simple flow channel detection system for rapid and sensitive analysis of bacterial cells. In a preferred embodiment, the system utilizes dye doped silica nanoparticles (NP) that provide highly luminescent signals and that can be easily used for bioconjugation with molecular probes for bioanalysis. The use of luminescent silica NPs not only provides significant signal amplification in bacterial antibody-antigen recognition, but also presents highly photostable luminescent signals for reproducible measurements.

The system of the subject invention is rapid, technically simple, highly sensitive and efficient. Using antibodies specific for various bacterial pathogens, this assay can be adapted for the detection of a wide variety of bacterial pathogens with high sensitivity, accuracy and fast speed.

One embodiment of the system of the present invention comprises an optical detection scheme that allows for the detection of the fluorescence signal of bacteria or other bioparticles in less than about 20 minutes. Advantageously, the microflow channel system of the subject invention allows for an assay probing volume of as little as a few picoliters.

In one embodiment, the system uses RuBpy dye-doped silica nanoparticles bioconjugated with specific monoclonal antibodies.

The system allows for the rapid and highly sensitive and specific detection of bacteria or other bioparticles without the need for amplification or enrichment of the sample.

### BRIEF DESCRIPTION OF THE DRAWINGS

FIG. 1 is a schematic diagram of one embodiment of a flow cytometer of the present invention.

FIG. 2 shows luminescence signals. FIG. 2a shows a typical luminescence burst recorded during the acquisition. FIG. 2b shows signals recorded when blank solution flows through the sample cell. FIGS. 2c and 2d show flow cytometry traces at different concentrations of bacteria samples.

FIG. 3 shows the number of O157 cells detected by flow cytometry counting vs. plating counting.

FIG. 4 shows a calibration curve of detection of bacterial cells using the flow cytometry system.

FIG. 5 shows a Gaussian probe volume containing cylindrical and curved volume contributions.

FIG. 6 is a diagram showing a reverse microemulsion procedure for nanoparticle synthesis.

FIG. 7 shows scanning electron microscope images. FIG. 7a shows nanoparticles for bioconjugation with antibodies for bacterium recognition; FIG. 7b shows an E. coli O157: H7 bacterium cell conjugated with antibody immobilized nanoparticles; FIG. 7c shows E. coli O157: DH-α; no nanoparticles are attached to the bacterium due to the lack of antigen for E. coli O157: H7 antibody.

FIG. 8 shows photostability results for RuBpy dye, RuBpy dye doped nanoparticles and post-coated RuBpy doped nanoparticles.

FIG. 9 shows photostability results for 1 μM TMR-Dextran dye.

### DETAILED DISCLOSURE OF THE INVENTION

The subject invention provides simple flow channel systems for the rapid detection of bacterial cells. In one embodiment, the system is capable of detecting single cells without enrichment. Advantageously, the system of the subject invention can be portable. Specifically exemplified herein is a system that uses bioconjugated nanoparticles.

The flow channel detection system of the subject invention provides enhanced analytical sensitivity, convenience in operation and excellent capability to detect single bacterial cells within a few minutes. In one embodiment of the present system, an excitation light beam is tightly focused to the center portion of a microcapillary flow cell, thereby reducing a probe volume to a few picolitres and resulting in low background signals.

To achieve high sensitivity, bioconjugated nanoparticles can be used according to the subject invention for bioanalysis. Nanoparticles are especially useful because they are very small, inert, bright, and easily modified for conjugation. For signaling, each nanoparticle of the subject invention preferably contains tens of thousands of dye molecules encapsulated in a protective silica matrix. When excited by an external energy source, the fluorescent dyes emit photons (fluorescence) that are observable and detectable for both quantitative and qualitative analysis. The nanoscale size of the nanoparticles minimizes physical interference with the biological recognition events. The nature of silica particles enables the relatively easy modification of the surface for conjugation with various biomolecules for a wide range of applications in bioassay systems. The ability to prepare the nanoparticles with existing fluorophores provides a diversity of nanoparticles for various applications.

In certain embodiments of the subject invention, biconjugated nanoparticles are incorporated with biorecognition molecules such as antibodies. In one embodiment, specific monoclonal antibodies are immobilized onto the nanoparticle surface to form nanoparticle-antibody conjugates. The antibody-conjugated nanoparticles can readily and specifically identify a variety of bacteria through antibody-antigen interaction and recognition. The conjugates bind to the target bacteria when they recognize the antigen on a bacterium surface, providing a bright luminescent signal for the detection of individual cells.

For a bacterium, there are many surface antigens available for specific recognition by using antibody-conjugated nanoparticles. Therefore, thousands of nanoparticles can bind to each bacterium, each nanoparticle preferably containing thousands of dye molecules, thereby producing a greatly amplified signal.

In one embodiment, silica nanoparticles are used. The highly luminescent and photostable silica nanoparticles facilitate a high level of sensitivity, which reduces or eliminates the need for further target amplification or enrichment of the bacterial samples.

The total number of target bacteria is obtained by counting the number of positive spikes in the flow channel detection system. To confirm the accuracy of this method, the average numbers of bacteria cells detected by the flow system were compared to those determined by a plate counting method. The two results correlated well. The combination of the flow system with the bioconjugated nanoparticles is highly sensitive, simple to use, portable and reproducible, and has excellent specificity for the detection of bacteria in various samples. The system can also be used to target other biological matter, such as DNA, mRNA, proteins, antigens and antibodies, for example.

As noted above, to improve the analytical sensitivity and to further reduce the time for detection of bacteria, one embodiment of the luminescence flow channel detection system of the subject invention uses a flow cytometer with bioconjugated nanoparticles for signal amplification. While there are many different types of nanomaterials for bioanalysis, one embodiment of the present invention uses luminophore doped silica nanoparticles (NPs). These NPs have unique and advantageous features such as intense luminescent signal, excellent photostability, and easy bioconjugation for linkage between nanomaterials and biological molecules for biological interactions and recognition. In addition, these NPs can be easily prepared and their surfaces can be modified with desired surface properties in both charge and functionality aspects.

In one embodiment, the signal enhancement of luminescent NPs is based on tens of thousands of luminescent dye molecules contained in a single NP, which forms the foundation for luminescence detection with significant optical signal amplification. Thus, the recognition of one binding site on the target, such as an antigen on a bacterium surface, is signaled by one NP instead of one dye molecule. Thus, the luminescent signals are tens of thousands of times higher than that provided by a single dye molecule, providing a highly amplified signal for single bacterium samples.

In a specific embodiment, the NPs are treated by immobilizing monoclonal antibodies that specifically bind to E. coli O157:H7 surface antigens for the recognition of the specific bacteria.

Nanoparticles with antibodies specific to other target particles immobilized at the nanoparticle surface can quantitate the presence of other pathogens and materials, including other bacteria, DNA, mRNA, proteins, antigens, antibodies and spores. Moreover, the system of the present invention can be used for the simultaneous detection of multiple materials, such as, for example, E. coli O157, S. typhimurium and B. cereus spores. In such multiplexed detection cases, different dyes can be used for multicolor analysis, for example.

Use of the NPs with the flow cytometry system of the present invention results in the accurate counting of bacterial cells based on the number of spikes assessed by the flow channel detection system. The combination of bioconjugated NPs and the portable flow cytometry system enables the detection of a single bacterium in a sample with fast speed, high sensitivity and excellent reproducibility.

### System Design

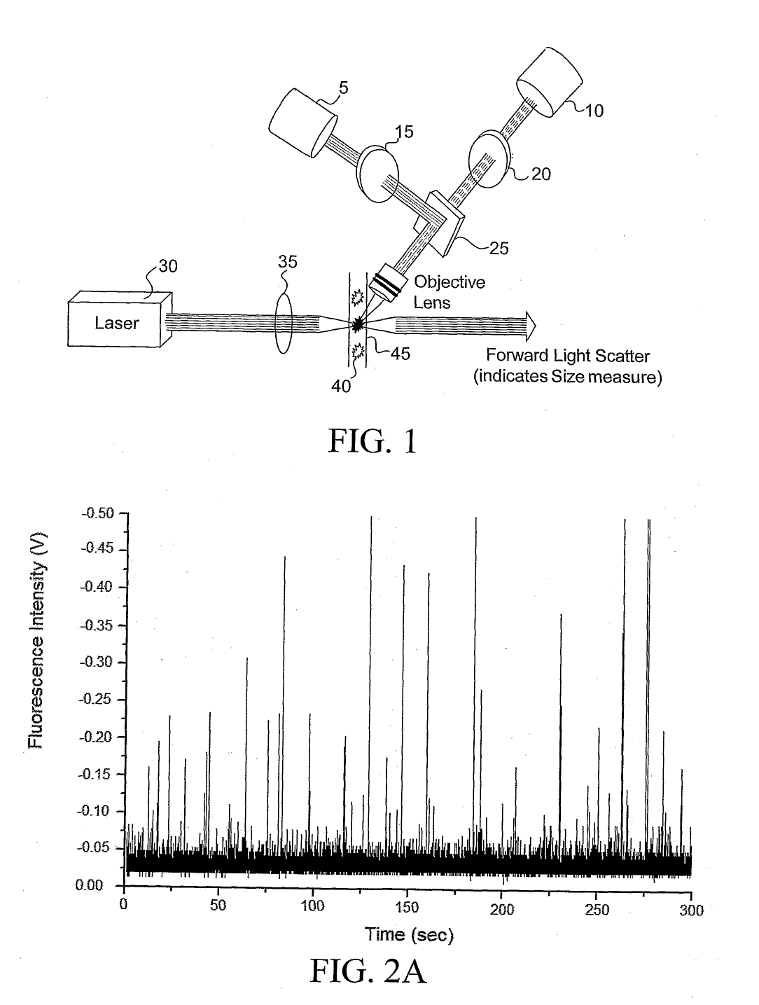

FIG. 1 is a schematic diagram of one embodiment of a portable flow cytometer device of the present invention. At least one photomultiplier tube is provided in a portable flow cytometer device, preferably at least two. Photomultiplier tubes (PMT1 and PMT2) 5, 10, as provided in a device of the invention, can contain built-in amplifier systems. At least one long pass filter is also provided in a portable flow cytometer device of the invention, preferably at least two. In a preferred embodiment, two long pass filters (F1 and F2) 15, 20 are provided, with F1, 15 at 570 nm and F2, 20 at 650 nm. An optical beam splitter (BS) 25 can also be provided in a portable flow cytometer device of the invention. A laser 30 for radiating light on the biomolecules present in a sample flowing through a flow cell (see below for details regarding the flow cell) is provided in the flow cytometer device of the invention. In certain embodiments a lens (L) 35 is provided, through which the light from the laser radiates. The lens 35 preferably focuses the light onto the biomolecules moving through the flow channel.

In one embodiment, an Argon (Ar⁺) laser from Omnichrome is used as the excitation source (488 nm). The laser beam is focused into the central region of a flow channel (see below for additional details) to probe the biomolecules present in a sample (such as bacterial species conjugated with the NPs). The ultrasensitive optical detection scheme is designed to detect the fluorescence signal as each bacterium passes through the probing volume. The luminescence emission is collected by a high numerical aperture (NA) microscope objective lens (40×, NA 0.65) placed at about 90° to the excitation and sample flow axes. Light transmitted is passed through a long pass (LP4951 nm) filter system (F1 and F2; 15, 20) to reduce scattered excitation. Luminescence bursts are detected with highly sensitive photomultiplier tubes (PMT1 and PMT2; 5, 10) containing built-in amplifier systems, available from Hammamatsu, Middlesex, N.J. In one embodiment, filter systems are disposed in front of each PMT to eliminate Raman and Rayleigh scattering which fall on the detectors. The bursts of luminescence from each biomolecule (i.e., bacterial species) are recorded through a 12-bit data acquisition card (NIDAQPad-6020E) interfaced to a laptop computer, and are then analyzed with a custom-built software (LabVIEW). The optical arrangement can be modified for the detection of several different biological species simultaneously.

In one embodiment, a sample flow cell is provided, through which biomolecules 40 present in a sample flow substantially one at a time in a straight line through a flow channel 45. The flow channel 45 in the optical flow cytometer device of the invention is preferably a silica microcapillary, such as one provided from Polymicro Technologies (Phoenix, Ariz.). The inner and outer diameters of the tube are 51 μm and 358 μm, respectively. In one embodiment, the sample flows at a constant rate through the micrometer-sized capillary/flow channel. An excitation and collection window (˜2 mm in length) is made by burning off the protective polymide sheath of the tube. The microcapillary is then fixed on an XYZ translator stage (Newport). In certain embodiments, samples are pumped through the capillary using a 1 ml syringe (Becton Dickinson, N.J.) and a mechanical microliter syringe pump (KdScientific). This arrangement provides a steady flow of samples through the channel at different flow rates, including from 1 μL/hr to 2 mL/hr. In one embodiment, the whole system is assembled inside a portable box, and the size of the system elements can be further reduced if needed.

Selection of Positive Signals from Background in the Flow Channel System

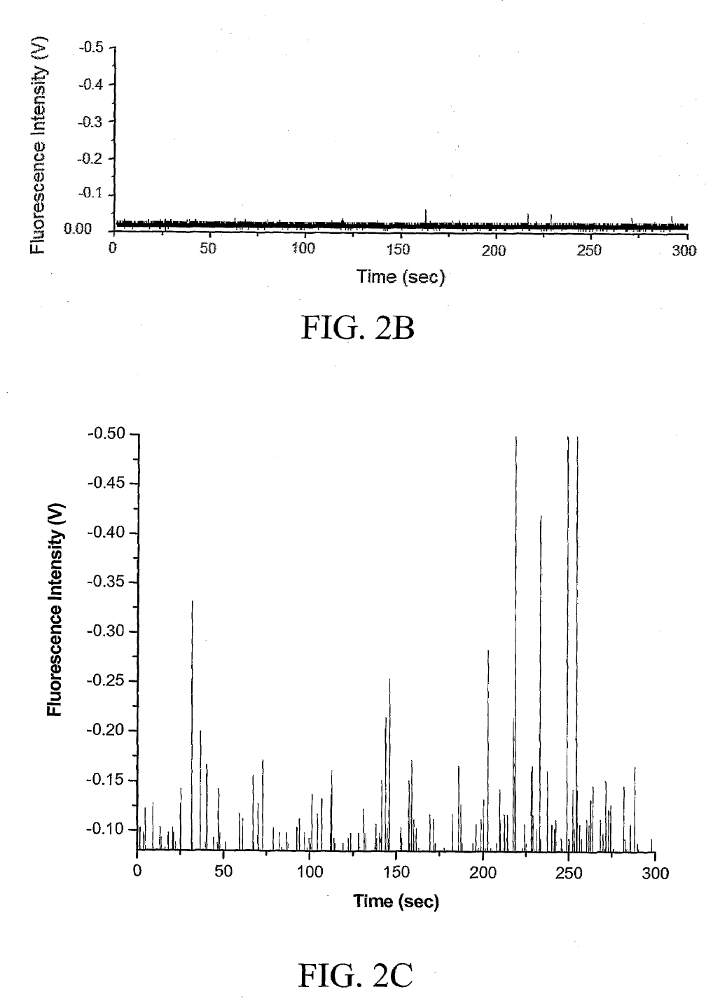

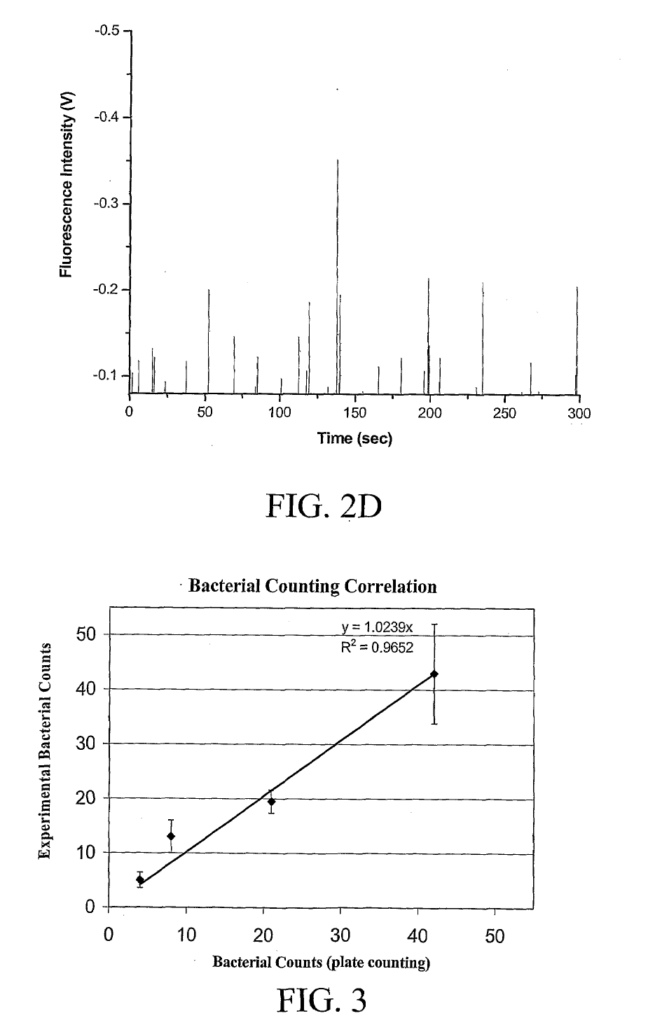

The emitted fluorescence signal during the passage of each bacterial cell through the probe volume represents an ‘event’ of the assay. The signals are detected by the PMT and acquired via a computer in real time. The recorded fluorescence data is an ensemble of positive spikes embedded along with background noise. FIG. 2a shows a typical luminescence burst recorded during the acquisition when a sample of bacteria and NP conjugates flows through the detection channel. FIG. 2b shows signals recorded when blank solution flows through the sample cell. In one embodiment, a threshold level (average signal intensity plus three times the blank sample's standard deviation) is set to discriminate the background noise from the signal. A spike that is higher than the threshold level represents one bacterial cell. Therefore, counting the number of spikes above the threshold level gives the number of the target bacteria. FIGS. 2c and 2d show fluorescence events above the threshold level for bacterial samples having concentrations of 5×10⁵ cells/mL and 1×10⁵ cells/mL, respectively. The current system is able to detect as few as one bacterium at a time. The sample flow speed and the sampling rates are adjustable according to the requirements of the different samples and the requirements of the analysis.

To evaluate the accuracy of the system of the invention in estimating bacterial counts, the average numbers of colony forming units (CFUs) of E. coli O157: H7 are determined by plate counting. Plate counting numbers are accepted as the standard in microbiology and are compared with the average numbers of E. coli O157: H7 detected by counting luminescent spikes in the flow cytometry analysis. In one example of accuracy evaluation, freshly cultured homogenate E. coli O157: H7 are grown in agar growth media for over 24 hours, and then the colonies are counted manually through visualization. The amounts of bacteria in the samples are also detected by an embodiment of the cytometry system of the present invention. FIG. 3 shows the number of O157 cells detected by plating counting vs. flow cytometry counting. It can be seen that the results obtained with these two methods correlate well. It is worthy to note that it is not uncommon that there is about 20% standard deviation in bacterium counting when using the plate counting method. The results show that the present invention has an accuracy comparable to the plate counting method but requires a much shorter analysis time.

### Quantitative Determination of Bacterial Cells Based on Counting Luminescent Spikes

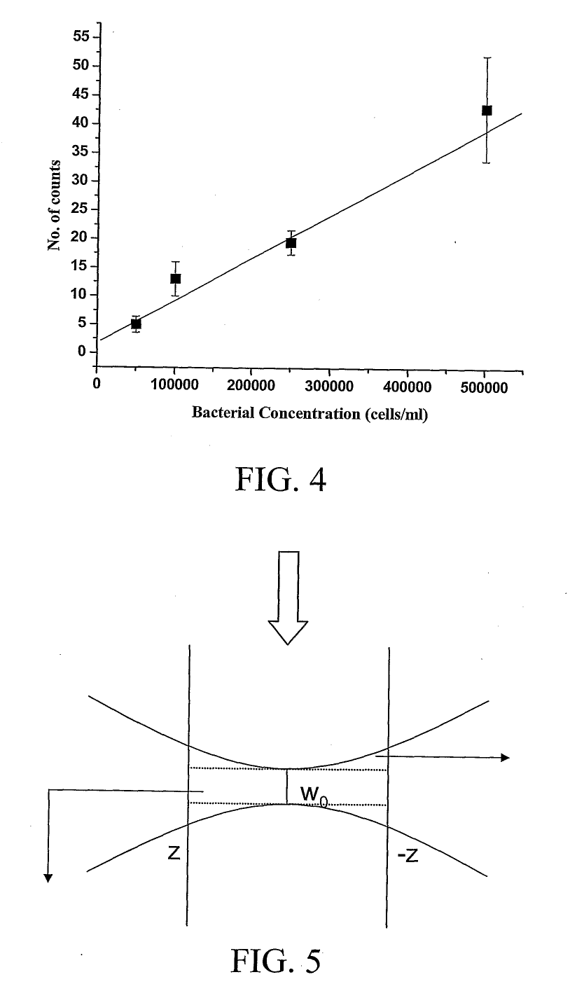

In one example, the system of the present invention is used to determine different concentrations of bacterial samples ranging from about 5×10⁴ cells/mL to about 5×10⁵ cells/mL at a flow rate of about 1 μL/hr. The number of spikes on the flow cytometry graph increases as the concentration of the bacteria sample increases. FIG. 4 shows a calibration curve based on these results.

Further investigation of the accuracy of the measurements is conducted with false-positive and false-negative tests. First, several blank samples are introduced into the detection system to determine if there is any false-positive signal. Then, several standard bacteria solutions in the same concentration are examined. Any failure to detect the presence of the targets gives a false-negative signal. The results showed that false-negative signals are insignificant and thus can be ignored. Meanwhile, several false-positive signals were observed when the microchannel and sample cell were washed ineffectively. To minimize the false positive signals, a washing solution containing 1M NaOH and 1% Tween 20 is employed to clean the microcapillary until false positives disappear, before conducting the next round of analysis.

In one example the total sample analysis time using the flow cytometry system of the present invention is less than about 10 minutes. In one embodiment, the flow rates are automatically controlled by the syringe pump system, and thus the detection time can be varied based upon the flow rate. The flow rates can be changed based on the bacterium concentration in the sample. The sampling rates can thus be very high.

In one example, a non-uniform burst size distribution is observed in the recorded luminescence data. FIG. 2 reveals that intensities of the spikes are not uniform. Such a distribution may result from, for example, non-uniform labeling of NPs onto the bacteria surface or varied luminescence detection by bacterial species flowing through the channel as a result of the bacterial species transiting the probe region by varied paths. Moreover, variations may be due to the rotation of the rod-shaped bacterial cells to different angles during the transit through the probe region. One way to avoid this problem is to conduct multiple experiments with both bacterium samples and control samples to set the threshold in such a way to minimize both false negative and false positive results.

The results shown in FIG. 3 and results from control experiments both suggest that the determination of bacterium samples is accurate. Non-uniform spike size is thus not a major problem as long as the size of the spike generated by the bacterium-NP conjugates is higher than the threshold. This is one important advantage of using NPs for this analysis as they provide a high signal upon binding on bacterium surfaces.

### Effect of the Probe Volume on Sensitivity in the Flow Cytometry

To sensitively detect trace amounts of targets, it is desirable to reduce background luminescence signals. A signal from a single molecule or a particle is independent of the probe volume while a background signal is proportional to the probe volume. Hence, the background signal can be minimized by using small probe volumes. FIG. 5 shows the probe volume, which has a Gaussian Profile when a laser beam is used. Here, ‘w₀’ is the beam waist of the focused Gaussian beam in the capillary tube. The probe volume consists of two regions with the central cylindrical region surrounded with a curved region.

Probe volume can be reduced by minimizing either the collimated beam radius or the collimating lens focal length. Test results show that smaller probe volumes lead to better signal to noise ratios for the detection of luminescent signals. According to the subject invention, the portable flow assay device of the invention sensitively detects and/or quantifies target biomolecules present in sample volumes from about 1 picoliter to 100 picoliters. Preferably, the sample volumes range from about 1 picoliter to 30 picoliters. In one example, the probe volume is reduced to about 14 picoliters (pL) by tightly focusing the excitation light beam to the center portion of the microcapillary sample cell.

### Reagents

Tetraethylorthosilicate (TEOS), triton X-100, tris(2,2′-bipyridyl) dichlororuthenium (II) hexahydrate RuBpy), succinic anhydride, morpholineethanesulfonic acid (MES), bovine serum albumin (BSA), 1-ethyl-3-3(3-dimethylaminopropyl) carbodiimide hydrochloride (EDC), and N-hydroxy-succinimide (NHS) are available from Sigma-Aldrich Chemical Co. Inc. Polycarbonate membranes (hydrophilic, 0.05 μm, 0.2 μm, 0.4 μm, and 0.8 μm), ammonium hydroxide (28-30 wt %), and all other chemicals of analytical reagent grade are available from Fisher Scientific Co. Monoclonal antibodies against E. coli O157:H7 are available from Biodesign International. E. coli O157:H7 and E. coli DH5α are available from American Type Culture Collections (ATCC). Distilled, deionized water (Easy Pure LF, Barnstead Co.) is used in the preparation of all aqueous solutions.

Following are examples which illustrate procedures for practicing the invention. These examples should not be construed as limiting. All percentages are by weight and all solvent mixture proportions are by volume unless otherwise noted.

### EXAMPLE 1

### Synthesis of Dye-Doped Silica Nanoparticles

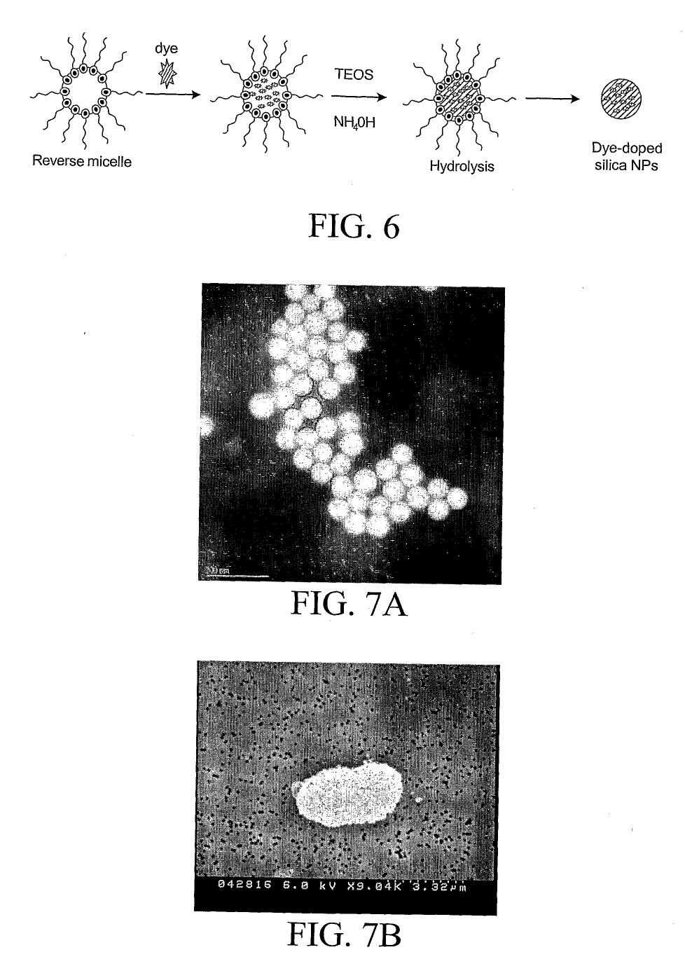

FIG. 6 is a diagram showing a reverse microemulsion procedure for nanoparticle synthesis. In one embodiment, using a reverse microemulsion method (also known as water-in-oil microemulsion), generally uniformly sized 60±4 nm spherical RuBpy-doped silica NPs are synthesized and characterized with respect to uniformity and luminescence properties. With a water-to-surfactant molar ratio (W₀) of 10, a reverse microemulsion is prepared by mixing about 7.5 mL cyclohexane, about 1.8 mL n-hexanol, about 1.77 mL triton x-100, about 80 μl of 0.01 M RuBpy, and about 400 μl water, followed by continuous stirring for about 20 minutes at room temperature. The size of the nanoparticles can be manipulated, as needed, by changing the water-to-surfactant molar ratio.

After adding about 100 μL of TEOS and about 60 μl of NH₄OH solution, which initiates the polymerization of Si(OH)₄ generated from the hydrolysis of TEOS, the reaction proceeds with continuous stirring for about 24 hours. When dye molecules are added to the microemulsion, they are trapped inside the silica matrix during polymerization. Fluorescence intensity is not necessarily proportional to dye concentration because dye loading beyond an optimal concentration may increase the occurrence of self-quenching. Carboxylated nanoparticles are directly produced by adding a carboxylated siliane during the postcoating of silica nanoparticles. About 25 μL of carboxylated siliane, N-(trimethoxysilylpropyl)-ethylenediamine, is added to the microemulsion to post coat the silica NPs. The NPs are released from the micelles with acetone and thoroughly washed with 95% ethanol. Ultrasonication and vortexing are used frequently during the washing steps to remove physically adsorbed residual reagents from the NP surface. The dye-doped NPs are air-dried and then stored at room temperature. Microemulsion desirably results in uniform dye-doped NPs having luminescent material dispersed throughout the silica sphere.

### EXAMPLE 2

### Immobilization of the Monoclonal Antibodies onto the Silica Nanoparticle Surface

The surface of the nanoparticle serves as a universal biocompatible and versatile substrate for the immobilization of biomolecules. In one embodiment of the present invention, after a thorough water wash, the silica surfaces of the RuBpy-doped carboxylated nanoparticles are activated, using about 100 mg/ml of 1-ethyl-3-3(3-dimethylaminopropyl) carbodiimide hydrochloride (EDC) and about 5 ml of 100 mg/ml N-hydroxy-succinimide (NHS) in a Z-morpholinoethanesulfonic acid (Mes) buffer (pH 6.8), for about 25 minutes at room temperature with continuous stirring. Water-washed particles are dispersed in about 10 ml of 0.1M PBS (pH 7.3) and reacted with monoclonal antibodies (mAbs) against E. coli O157: H7 for about 3 hours at room temperature with continuous stirring. To covalently immobilize the monoclonal antibodies onto the NP surface, about 5 ml of 0.1 mg/ml nanoparticles is reacted with about 2 ml of 5 μg/ml antibody for E. coli O157 for about 2 to about 4 hours at room temperature with continuous stirring. In one embodiment, the resultant antibody-conjugated nanoparticles are washed with a PBS buffer.

To reduce the effects of nonspecific binding in the subsequent immunoassay, the antibody-conjugate nanoparticles are reacted with 1% BSA and washed in 0.1M PBS (pH 7.3) before being used in the immunoassay. With storage at 4° C., the chemically modified RuBpy-doped silica-coated NPs are viable for several months, while the reporter antibodies are active for up to about two weeks. If the NP-antibody conjugates are stored at −20° C., they are stable for several months.

### EXAMPLE 3

### Detection of Bacteria

A 500 μL bacterial sample, which contains 25 bacteria based on plate-counting results, is dispersed into about 500 μL of 0.1 mg/ml of antibody conjugated NPs in a 0.1 M PBS buffer (pH 7.3) for about ten minutes. To remove the free antibody conjugated NPs that did not bind to the bacteria, the samples are centrifuged at about 14,000 rpm for about 30 seconds, and then the supernatant is removed. The samples are washed again to remove all unbound antibody conjugated NPs, and about 1.0 ml of PBS buffer is added to the samples. Samples are pumped through the capillary using a 1 ml syringe and a mechanical microliter syringe pump. This allows for a steady flow of sample through the channel at controllable various flow rates, including, for example, sample flow rates ranging from about 1 μL/hr to about 2 mL/hr. In another embodiment, control samples are obtained using the same experimental procedures but without the addition of bacteria.

### EXAMPLE 4

### Bioconjugated Luminescent Nanoparticles for Bacterium Recognition

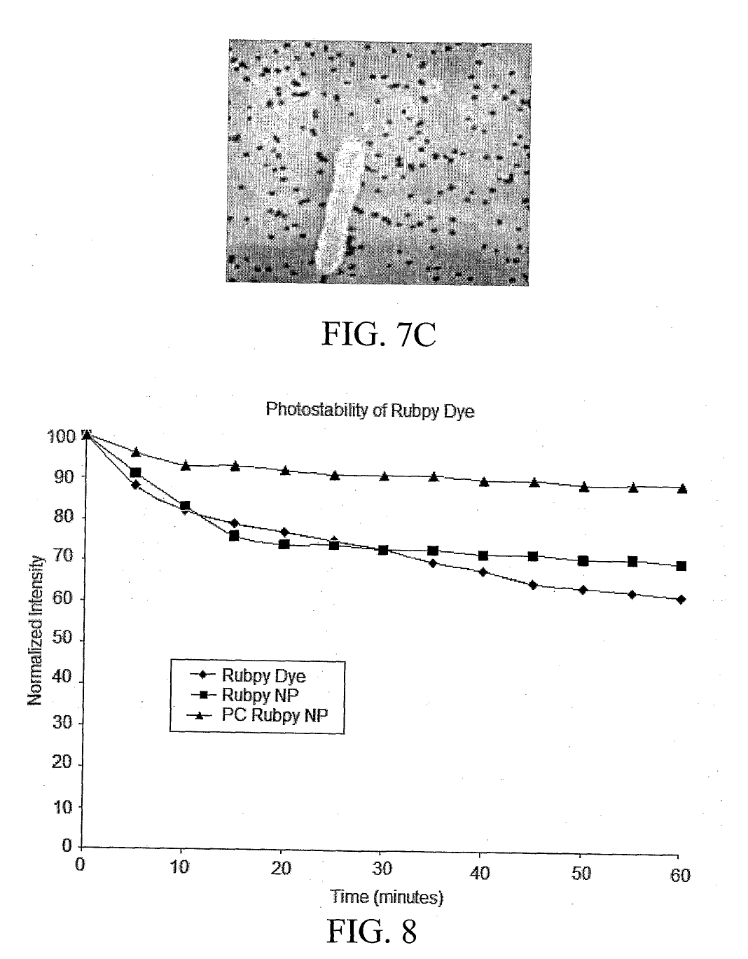

The luminescent NPs are prepared with 60±4 nm NPs in one embodiment of the invention. There are tens of thousands of dye molecules encapsulated within each NP. The antibody conjugated NPs are then used for the recognition of bacterium. The monoclonal antibody immobilized on the NPs is highly selective for E. coli O157:H7 in the immunoassay. Therefore, the antibody conjugated NPs specifically associate only with E. coli O157:H7 cell surfaces (FIG. 7a), but not with E. coli DH5α, for example, which lacks the surface O157:H7 antigen (FIG. 7b).

The scanning electron microscope (SEM) image of FIG. 7c of the E. coli O157:H7 cell following incubation with the NPs shows that there are many antibody-conjugated NPs bound to a single bacterium, providing significant luminescent signal amplification as compared to a single dye molecule assay.

The greatly amplified and photostable luminescent signals from NPs labeled onto the bacteria surface enables the easy distinction of the spikes of the bacteria from the background. The luminescence intensity of one RuBpy-doped NP is equivalent to that of more than 10⁴RuBpy molecules. The highly luminescent signal is particularly important when only one bacterium or just a few bacteria exists in a sample or when there is a high level of background luminescence. Moreover, since there is no significant wavelength shift, dye doped nanoparticles provide essentially the same excitation and emission characteristics as free dyes.

When monoclonal antibodies are immobilized onto the NPs for the immunoassay, the presence of the NPs does not appreciably reduce the affinity of the antibody to the antigen. Moreover, the affinity constants may be slightly higher than the intrinsic affinity of the antibody. The NP-antibody conjugates on the bacterium surface show a strong binding affinity to E. coli O157: H7 cells and thus give very bright luminescent signals.

Table 1 below shows exemplary sizes and functionalities of RuBpy doped and TMR-Dextran doped nanoparticles.

TABLE 1

| Fluorescent Dye | Nanoparticle Modification | Size (nm) |
|---|---|---|
| Rubpy Doped NP | No Post-Coating | 68 ± 4 |
| | Post-Coated | 95 ± 7 |
| TMR-Dextran Doped NP | No Post-Coating | 76 ± 5 |
| | Post-Coated | 101 ± 6 |
| | Phosphate Modified | 105 ± 8 |
| | Carboxylic Acid Modified | 98 ± 5 |
| | C-12 Modified | 107 ± 8 |
| | PEG Modified | 113 ± 11 |
| | NH₂ Modified | 103 ± 7 |
| | Stober Post-Coat | 126 ± 3 |
| | Stober Particles | 88 ± 6 |

### EXAMPLE 5

### Photostability

The luminescence signals provided by the NPs are not only very bright but also reproducible due to greatly reduced photobleaching, even under continuous excitation. Because of the protective function of the silica matrix and post coat, the nanoparticles are highly photostable. This high photostability provides reliable testing measurements. The NPs are thus unique in providing reproducible and highly amplified signals for biorecognition.

FIG. 8 shows photostability results for RuBpy dye, RuBpy dye doped nanoparticles and post-coated RuBpy doped nanoparticles. After about 60 minutes of continuous laser excitation, observations using a fluorescence microscope show that RuBpy dye fluorescence decreases in intensity about 38%, RuBpy dye-doped nanoparticle fluorescence decreases about 30%, and post-coated RuBpy dye-doped nanoparticles maintained about 90% of their initial intensity. Thus, adding a post coating of silica to the particles enhances particle photostability. The increased photostability observed is due to the enhanced protection of the dye molecules from the outside environment by the silica matrix.

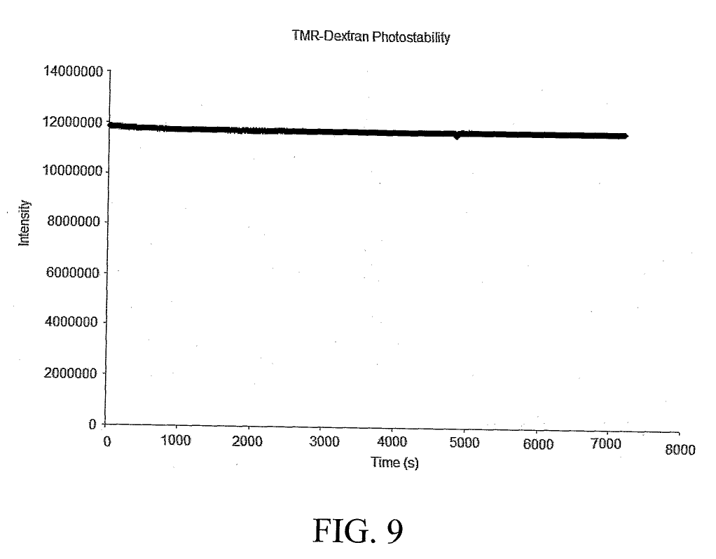

FIG. 9 shows photostability results for 1 μM TMR-Dextran dye. In a photostability test, the fluorescence intensity for TMR-Dextran appears to remain constant for a period of about 2 hours. The resistance to photobleaching leads to the conclusion that TMR-Dextran is not a photosensitive compound. Compared to TMR dye (with no dextran linker), the dextran linked dye is much more photostable.

TMR-Dextran dye doped particles made by the microemulsion and Stöber methods were also tested. The intensity of all samples tested remained constant for a period of about 2 hours. The post-coated particles with numerous functional groups were also tested for 2 hours with no significant changes in intensity. These results show that TMR-Dextran dye is a photostable, water soluble fluorophore. Therefore, TMR-Dextran doped silica nanoparticles will also be resistant to photobleaching. The enhanced resistance to photodegradation and large quantum yield make TMR-Dextran an especially suitable fluorophore for bioanalysis. Because TMR-Dextran is a photostable fluorescent molecule, it is not necessary to dope the TMR-Dextran dye into a silica nanoparticle or to post-coat the nanoparticle to prevent photobleaching. However, such doping does offer the benefits of stronger signals, easy surface modification, and lack of toxicity effects.

### EXAMPLE 6

### Nanoparticles with Amine-Functionalized Groups

To form amine-functionalized groups on the nanoparticle surfaces, about 32 mg of silica nanoparticles are reacted with about 20 ml of 1% trimethoxysilyl-propyldiethylenetriamine in 1 mM acetic acid for about 30 minutes at room temperature, with continuous stirring. These nanoparticles are thoroughly washed about three times in distilled, deionized water. After washing with N,N-dimethylformamide, the nanoparticles are reacted with 10% succinic anhydride in N,N-dimethylformamide solution under N₂ as for about 6 hours with continuous stirring. Thus, functional groups are formed onto the silica nanoparticle surface for conjugation of antibodies.

### EXAMPLE 7

### Nanoparticle Activated with Functional Groups

In another embodiment, the reverse microemulsion procedure combines about 1.77 mL Triton x-100, about 7.5 mL cyclohexane, about 1.6 mL n-hexanol, and about 480 μL of 1 mM Tetraethylrhodamine-Dextran (TMR-Dextran, MW=3000, anionic, available from Molecular Probes, Inc.) in HCl solution (pH=1.5), followed by the addition of 100 μL tetraorthosilicate (TEOS), and 60 μL NH₄OH to initialize the silica polymerization reaction. After 24 hours, the microemulsion is broken by overwhelming the solution with ethanol, and then the solution is centrifuged, sonicated, vortexed, and washed with 95% ethanol four times, followed by one wash with H₂O. The post-coating procedure involves re-dispersing the particles in a microemulsion solution of ethanol and NH₄OH and allowing the reaction to proceed for 24 hours with an additional amount of TEOS. The introduction of reactive chemicals to the microemulsion during the post coating procedure renders the surface of the particles activated with various functional groups. Washing procedures are repeated to collect the post-silica coated nanoparticles and surface functionalized post-silica coated nanoparticles.

The reverse microemulsion is a thermodynamically stable process consisting of a surfactant (Triton x-100), oil (cyclohexane), and water. The oil solvent contains nano-sized water droplets which act as the reactor for a Sol-Gel reaction. When dye molecules are added to the microemulsion, they are trapped inside the silica matrix during polymerization. For TMR-Dextran dye doped nanoparticles, acidic conditions can be used to create an electrostatic attraction between the silica and dye molecules. Varying microemulsion conditions—for example increasing the amount of surfactant or reaction time—allows for a large range of nanoparticle sizes to be synthesized.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A portable flow assay device for rapid detection and/or quantification of biomolecules in a sample having a volume of about 1 picoliter to about 100 picoliters, said device comprising:
(a) a flow cell for moving biomolecules substantially one at a time in a straight line through a flow channel;
(b) a laser for radiating light on the biomolecules moving through said flow cell;
(c) a lens for focusing the radiated light onto the biomolecules moving through said flow cell;
(d) an objective lens for detecting light radiated at a 90° angle with respect to the direction of the radiated light from the laser;
(e) an optical beam splitter;
(f) at least one long pass filter; and
(g) at least one photomultiplier tube.

2. The portable flow assay device of claim 1, comprising two photomultiplier tubes that contain built-in amplifier systems.

3. The portable flow assay device of claim 1, comprising first and second long pass filters, wherein the first filter is at 570 nm and the second filter is at 650 nm.

4. The portable flow assay device of claim 1, wherein the laser is an argon laser.

5. The portable flow assay device of claim 1, wherein the flow channel is composed of silica and has an inner diameter of about 51 μm and an outer diameter of about 358 μm.

6. The portable flow assay device of claim 1, further comprising an excitation and collection window about 2 mm in length.

7. The portable flow assay device of claim 1, further comprising a translator stage on which the flow channel is affixed.

8. The portable flow assay device of claim 1, further comprising a syringe through which the samples are pumped into the flow channel.

9. The portable flow assay device of claim 1, wherein the sample has a volume of about 1-30 picoliters.

10. A system for detecting or quantifying target pathogens or biomolecules comprising:
(a) at least one nanoparticle that is highly specific for at least one target pathogen or biomolecule, wherein said nanoparticle comprises a means for signaling binding of the nanoparticle to the target pathogen or biomolecule; and
(b) a flow channel assay.

11. The system of claim 10, wherein the nanoparticle further comprises at least one biorecognition molecule that enables the nanoparticle to be highly specific for the target pathogen or biomolecule.

12. The system of claim 11, wherein the biorecognition molecule is an antibody.

13. The system of claim 10, wherein the means for signaling comprises a plurality of dye molecules, wherein the at least one nanoparticle is a silica nanoparticle, and wherein the dye molecules are encapsulated in the silica nanoparticle.

14. The system of claim 13, wherein the dye molecules are luminescent.

15. The system of claim 10, wherein the flow channel assay is a flow cytometer.

16. The system of claim 1S, wherein the flow cytometer is portable.

17. The system of claim 10, wherein the target pathogen or biomolecule is selected from the group consisting of: bacteria, DNA, mRNA, proteins, antigens, antibodies, spores, and any combination thereof.

18. The system of claim 17, wherein a plurality of target pathogens or biomolecules are quantified or detected, wherein the target pathogens or biomolecules are selected from the group consisting of: E. coli O157, S. typhimurium spores, B. cereus spores, and any combination thereof.

19. The system of claim 10, further comprising a computing means for recording and presenting data regarding the detected or quantified target pathogens or biomolecules.

20. A method for detecting or quantifying target pathogens or biomolecules comprising:
(a) exposing a sample comprising at least one target pathogen or biomolecule to at least one nanoparticle that is highly specific for the target pathogen or biomolecule, wherein said nanoparticle comprises a means for signaling binding of the nanoparticle to the target pathogen or biomolecule;
(b) running the sample of step (a) through a flow assay device; and
(c) detecting or quantifying any signal activity from the signaling means;
wherein the flow assay device is a portable device for rapid detection and/or quantification of biomolecules in a sample having a volume of about 1 picoliter to about 100 picoliters, said device comprising:
(a) a flow cell for moving biomolecules substantially one at a time in a straight line through a flow channel;
(b) a laser for radiating light on the biomolecules moving through said flow cell;
(c) a lens for focusing the radiated light onto the biomolecules moving through said flow cell;
(d) an objective lens for detecting light radiated at a 90° angle with respect to the direction of the radiated light from the laser;
(e) an optical beam splitter;
(f) at least one long pass filter; and
(g) at least one photomultiplier tube.
